(19) United States
(12) Patent Application Publication
(10) Pub. No.: US 2022/0039832 A1
(43) Pub. Date: Feb. 10, 2022

(54) ULTRASONIC GUIDED PUNCTURE DEVICE AND ULTRASONIC GUIDED PUNCTURE APPARATUS

(71) Applicant: SHENZHEN INSTITUTES OF ADVANCED TECHNOLOGY, SHENZHEN (CN)

(72) Inventors: WEIBAO QIU, SHENZHEN (CN); MIN SU, SHENZHEN (CN); ZHIQIANG ZHANG, SHENZHEN (CN); RUILIN CAI, SHENZHEN (CN); HAIRONG ZHENG, SHENZHEN (CN)

(21) Appl. No.: 17/506,705

(22) Filed: Oct. 21, 2021

Related U.S. Application Data
(63) Continuation of application No. PCT/CN2019/104001, filed on Sep. 2, 2019.

(30) Foreign Application Priority Data
Apr. 23, 2019 (CN) 201910329546.4

(51) Int. Cl. A61B 17/34

(52) U.S. Cl. CPC A61B 17/3403; A61B 2017/3413; A61B 17/3472

(57) ABSTRACT

An ultrasound guided puncture device and an ultrasound guided puncture apparatus are disclosed. The ultrasound guided puncture device includes a puncture needle tube and an ultrasound transducer. One end of the puncture needle tube has a puncture needle head, and the ultrasonic transducer is housed in the puncture needle tube and extends to the puncture needle head. The ultrasonic transducer includes multiple ultrasonic array elements for transmitting and receiving ultrasonic waves, and the multiple ultrasonic array elements are arranged at the puncture needle head. In actual use, electronic scanning imaging is performed through the multiple ultrasound array elements, hence a wide imaging range. It can effectively recognize the structure of the tissue in front of the puncture needle head, thereby facilitating the planning of the best puncture path.

### CROSS-REFERENCE TO RELATED APPLICATIONS

This application is a U.S. continuation of co-pending International Patent Application Number PCT/CN2019/104001, filed Sep. 2, 2019, which claims the benefit and priority of Chinese Patent Application Number 201910329546.4, filed Apr. 23, 2019, before China National Intellectual Property Administration, the disclosures of which are incorporated herein by reference in their entireties.

### TECHNICAL FIELD

The present disclosure relates to the field of medical equipment, and more particularly relates to an ultrasound guided puncture device and an ultrasound guided puncture apparatus.

### BACKGROUND

Vertebral puncture technology has long been a technical problem in the field of medical applications. Due to the complex tissue environment near the human spine and lumbar vertebrae, various components including fat, muscles, ligaments, compact bones, and cartilages increase the difficulty of puncturing the vertebrae by medical staff. Thus, a real-time and effective method is needed to guide the medical staff to perform the puncture.

Compared with X-ray, CT, etc., ultrasound is a real-time, portable, less painful, and free of side effects imaging method. As such, ultrasound-guided vertebral puncture has attracted more and more attention. In the related art, the ultrasound guided puncture method consists in performing imaging during puncture by fixedly or non-fixedly mounting an array ultrasound transducer on the surface of the human skin to guide the advancing route of the hypodermic needle. However, this method is difficult to operate, and medical staff need to operate the array transducer and the needle at the same time. In addition, the components near the vertebrae are complex, and it is difficult to distinguish between the nearby tissue components from one another with an external transducer, and some cartilages and ligaments are relatively small, so that a relatively high-frequency transducer is required to ensure the imaging resolution. When puncturing an obese patient, it is difficult to ensure that an imaging array transducer placed external to the body has a sufficient imaging depth.

In view of this, it is particularly important to design and manufacture an ultrasound guided puncture device that has good imaging effect, is capable of simple operation, can distinguish between the structures and morphologies of the tissues ahead of the needle, and can plan the best puncture route for the needle tube.

### SUMMARY

It is there an object of the present disclosure to provide an ultrasound guided puncture device that has good imaging effect, is capable of simple operation, can distinguish between the structures and morphologies of the tissues ahead of the needle, and can plan the best puncture route of the needle tube.

It is yet another object of the present disclosure to provide an ultrasound guided puncture apparatus, which can control the imaging range, has good imaging effect and is capable of simple operation.

The present disclosure is implemented by adopting the following technical solutions.

There is provided an ultrasound guided puncture device that includes a puncture needle tube and an ultrasonic transducer. One end of the puncture needle tube has a puncture needle head. The ultrasonic transducer is housed in the puncture needle tube and extends to the puncture needle head. The ultrasonic transducer includes a plurality of ultrasonic array elements configured for transmitting and receiving ultrasonic waves, which are arranged at the puncture needle head.

Further, the puncture needle head is pointed and has a containing head, and the plurality of ultrasonic array elements are arranged in the containing head.

Further, the puncture needle head has a needle tip side and a needle tail side which are opposite to each other, and the plurality of the ultrasonic array elements are arranged between the needle tip side and the needle tail side.

Further, the plurality of ultrasonic array elements are distributed in an array.

Further, the plurality of ultrasonic array elements are distributed horizontally.

Further, the plurality of ultrasonic array elements are distributed in a staircase shape, and there is a height difference between the ultrasonic array elements distributed along the direction of the steps.

Further, the end surface of the puncture needle head has a puncture bevel, and the array composed of the plurality of ultrasonic array elements has an array bevel that matches the puncture bevel.

Further, the ultrasonic transducer further includes a flexible member and a tubular housing. The tubular housing is accommodated in the puncture needle tube and extends to the puncture needle head. The flexible member is accommodated in the tubular housing. The plurality of ultrasonic array elements are arranged at one end of the flexible member adjacent to the puncture needle head, and the plurality of ultrasonic array elements are all electrically connected to the flexible member.

Further, the end of the tubular housing has a puncture head, which is accommodated in the puncture needle head. The end surface of the puncture head and the end surface of the puncture needle head are arranged flush or at an angle.

Further, the plurality of the ultrasonic array elements are arranged in the tubular housing.

There is further provided an ultrasound guided puncture apparatus that includes an ultrasound electronic system and an ultrasound guided puncture device. The ultrasound guided puncture device includes a puncture needle tube and an ultrasonic transducer. One end of the puncture needle tube has a puncture needle head. The ultrasonic transducer is housed in the puncture needle tube and extends to the puncture needle head. The ultrasonic transducer includes a plurality of ultrasonic array elements configured for transmitting and receiving ultrasonic waves, which are arranged at the puncture needle head. The ultrasonic electronic system is electrically connected to the ultrasonic transducer.

This disclosure may have the following beneficial effects.

In the ultrasound guided puncture device provided by the present disclosure, the ultrasonic transducer is arranged in the puncture needle tube and extends to the puncture needle head, and ultrasonic waves are transmitted and received through a plurality of ultrasonic array elements arranged at the puncture needle head. In actual use, electronic scanning imaging can be performed based on the multiple ultrasound array elements, hence a wide imaging range. It can effectively distinguish between the morphologies of various tissues ahead of the puncture needle head, thereby facilitating the planning of the best puncture path. After the puncture, the ultrasound transducer can be pulled out from the puncture needle tube, and then anesthetic can be released from the puncture needle tube or body fluids can be extracted. Compared with the existing external array transducer, the ultrasound guided puncture device provided by the present disclosure is closer to the imaged tissue, so that the imaging is clearer. It is not easy to be obscured by other parts to affect the imaging, and the operation is convenient and does not require multiple medical staff to operate in cooperation.

### BRIEF DESCRIPTION OF DRAWINGS

FIG. 1 is a schematic diagram illustrating an ultrasound guided puncture device provided by a first embodiment of the present disclosure.

FIG. 2 is a schematic diagram illustrating the overall structure of the ultrasonic transducer shown in FIG. 1.

FIG. 3 is a sectional view of a partial structure of the ultrasonic transducer shown in FIG. 1.

FIG. 4 is a schematic diagram illustrating a partial structure of the ultrasonic transducer shown in FIG. 1.

FIG. 5 is a schematic diagram illustrating an ultrasonic transducer provided by a second embodiment of the present disclosure.

FIG. 6 is a schematic diagram illustrating a partial structure of an ultrasound guided puncture device provided by a third embodiment of the present disclosure.

FIG. 7 is a schematic diagram illustrating a partial structure of an ultrasound guided puncture device provided by a fourth embodiment of the present disclosure.

Reference signs used in the drawings: 100-Ultrasound guided puncture device; 110-Puncture needle tube; 111-Puncture needle head; 113-Needle tip side; 115-Needle tail side; 130-Ultrasonic transducer; 131-Ultrasonic array element; 1311-Matching layer; 1313-Piezoelectric Layer; 1315-Backing layer; 133-Flexible member; 135-Tubular housing; 150-Handle.

### DETAILED DESCRIPTION OF ILLUSTRATIVE EMBODIMENTS

For a better understanding of the objectives, technical solutions, and advantages of the present application, hereinafter the present application will be described in further detail in connection with the accompanying drawings and some illustrative embodiments. It is to be understood that the specific embodiments described here are intended for the mere purposes of illustrating this application, instead of limiting.

It should be noted that similar reference numerals and characters indicate similar items in the following drawings. Thus, once an item is defined in one drawing, it does not need to be further defined and explained in the subsequent drawings.

As used herein, terms “center”, “upper”, “vertical”, “horizontal”, “in”, “inside”, “inner”, “out of”, “outside”, “outer”, etc. are used to indicate orientational or relative positional relationships based on those illustrated in the drawings, or the orientational or positional relationship that the product of the invention is usually placed in use. They are merely intended for simplifying the description of the present disclosure, rather than indicating or implying that the device or element referred to must have a particular orientation or be constructed and operate in a particular orientation. Therefore, these terms are not to be construed as restricting the present disclosure. In addition, terms “first”, “second”, or the like are merely used for purposes of distinguishing, and are to be understood as indicating or implying relative importance.

Furthermore, as used herein, terms “disposed on”, “arranged on”, “connected to”, “coupled to”, “mounted on”, “installed on”, “connected with”, and “coupled with” should be understood in a broad sense unless otherwise specified and defined. For example, they may indicate a fixed connection, a detachable connection, or an integral connection. They may denote a mechanical connection, or an electrical connection. They may denote a direct connection, a connection through an intermediate, or an internal connection between two elements. For those of ordinary skill in the art, the specific meanings of the above terms as used in the present application can be understood depending on specific contexts.

Cerebrospinal fluid (CSF) is an important body fluid that can be used to diagnose various central nervous system diseases or disorders, including life-threatening diseases such as encephalitis or meningitis. Delays in the diagnosis of some diseases by several hours may cause serious consequences. Lumbar puncture (LP) is an effective method to obtain cerebrospinal fluid. At present, most lumbar punctures use anatomical landmarks to locate the intervertebral space between L3-L5, and then use a puncture needle to penetrate several tissue layers between the vertebrae to enter the subarachnoid space, without touching other obstacles (e.g., blood vessels or bones) along the way. Most lumbar punctures are performed “blindly” without the help of imaging or guidance mechanisms. Approximately 23.3% of the people that undergo lumbar puncture every year end up in failure. These errors lead to delays in treatment and create unnecessary and dangerous procedures. Some obese patients have too much adipose tissue between the epidermis and the target, so that the failure rate of lumbar puncture is even higher. The complication rate due to lumbar puncture has almost increased to 50% in obese patients.

Epidural injection is a new type of anesthesia technology that can effectively relieve lower body pain during surgery and childbirth, and can replace general anesthesia and so is currently very popular. During an epidural injection, a needle is inserted into the epidural space between the ligamentum flavum (LF) and the dura, and then a catheter is inserted to deliver the anesthetic. Nowadays, epidural injections mostly use the blind insertion method of loss of resistance (LOR) technique. According to this method, the anesthesiologist touches the patient's vertebrae through manual palpation, selects a suitable gap and inserts it along the midline of the spine. As for the depth of insertion, the anesthesiologist feels a constant resistance during the insertion of the needle tube, and when the needle tube is inserted into the epidural space, the resistance will become weaker so that the anesthesiologist would consider that the right position has been found. However, it is difficult to find a suitable insertion position for obese patients, and there are differences in tissue resistance between patients. Sometimes it cannot be felt at all, and even experienced anesthesiologists can be misled. Therefore, it often takes multiple punctures to accomplish the injection, which brings great pain to the patient, and experienced anesthetists and novice anesthetists will respectively have 1-3% and 3-5% probabilities of causing dural puncture complications during the epidural injection. Dural puncture can cause temporary or irreversible permanent complications, such as headache, epidural hematoma, or nerve damage. Thus, epidural injection is still one of the most challenging tasks performed by anesthesiologists, and a better method is needed to guide the anesthesiologist for accurate puncture.

As mentioned in the Background section, most of the existing puncture guidance methods use an external array ultrasound array transducer to perform imaging during puncture to guide the advancing route of the needle tube. However, this method is difficult to operate and the imaging effect is not good. Then there emerges a single-element transducer that is placed at the front end of the needle tube and that can receive the echoes reflected by the detected tissues in front. However, the information obtained is too abstract and singular so that it is difficult to determine the composition and morphologies of the tissues in front through individual echoes. Furthermore, due to the unreasonable installation structure of the single-element transducer, it is very difficult to remove, and it can only be used as a detection method, but cannot be used as a treatment method. The present disclosure provides an ultrasound guided puncture device, in which an ultrasound transducer containing multiple array elements are installed at the front end of a needle tube, thus realizing ultrasound electronic scanning imaging through the ultrasound transducer to obtain ultrasound images in the front side area of the transducer, thereby further distinguishing the structures and morphologies of the tissues in front of the needle tube, planning the best puncture route, and the operation is very convenient, without need multiple people to operate.

Hereinafter, some embodiments of the present application will be described in detail with reference to the accompanying drawings. In the case of no conflict or contradiction, the features described in the following embodiments may be combined with each other.

### First Embodiment

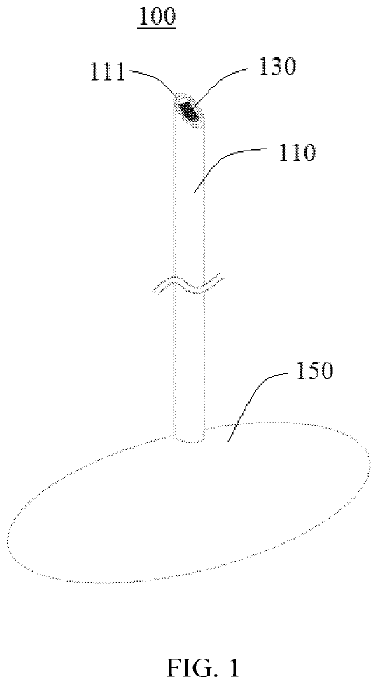

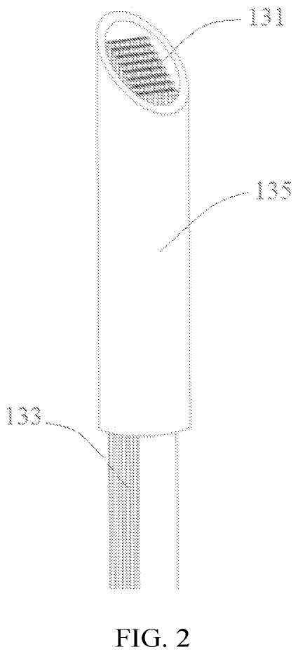

With reference to FIGS. 1 and 2 in conjunction, this embodiment provides an ultrasound guided puncture device 100, including a puncture needle tube 110 and an ultrasound transducer 130. The puncture needle tube 110 has a puncture needle head 111 at one end, and the ultrasonic transducer 130 is accommodated in the puncture needle tube 110 and extends to the puncture needle head 111. The ultrasonic transducer 130 includes a plurality of ultrasonic array elements 131 for transmitting and receiving ultrasonic waves, and are arranged at the puncture needle head 111.

In actual use, electronic scanning imaging can be performed through the multiple ultrasound array elements 131, hence a wide imaging range. It can effectively recognize the morphologies of various tissues ahead of the puncture needle head 111, thereby facilitating the planning of the best puncture path. After the puncture, the ultrasound transducer 130 can be pulled out from the puncture needle tube 110, and then anesthetic can be released from the puncture needle tube 110 or body fluids can be extracted through the puncture needle tube 110, which is very convenient to operate.

In this embodiment, the other end of the puncture needle tube 110 is installed with a handle 150, which is connected to the end of the ultrasonic transducer 130 facing away from the puncture needle head 111.

Further, the ultrasonic transducer 130 further includes a flexible member 133 and a tubular housing 135. The tubular housing 135 is housed in the puncture needle tube 110 and extends to the puncture needle head 111. The flexible member 133 is housed in the tubular housing 135. The plurality of ultrasonic array elements 131 are arranged at one end of the flexible member 133 adjacent to the puncture needle head 111, and the plurality ultrasonic array elements 131 are all electrically connected to the flexible member 133. In particular, the bottom of each ultrasonic array element 131 may be provided with an electrode, and the electrodes of the plurality of ultrasonic array elements 131 may be led out through the flexible member 133 to facilitate electrical connection with other external control devices.

In this embodiment, the flexible member 133 may be a flexible circuit board or a flexible cable, but any conductive structure capable of achieving flexible connection shall fall in the scope of protection of the present disclosure.

It should be noted that in this embodiment, the tubular housing 135 is fitted in the puncture needle tube 110, and the tubular housing 135 can withdraw outward along the puncture needle tube 110 under the action of an external force, which facilitates the use of the puncture needle tube 110 to perform treatment operations after the puncture reaches the desired position.

In this embodiment, the end of the tubular housing 135 has a puncture head, which is accommodated in the puncture needle head 111, and the end surface of the puncture head is flush with the end surface of the puncture needle head 111. In particular, the tubular housing 135 has a puncture head, which has a pointed shape to facilitate the puncture. In other exemplary embodiments of the present disclosure, the tubular housing 135 may also be retracted within the puncture head without participating in the puncture action, but the specific structure thereof will not be described herein.

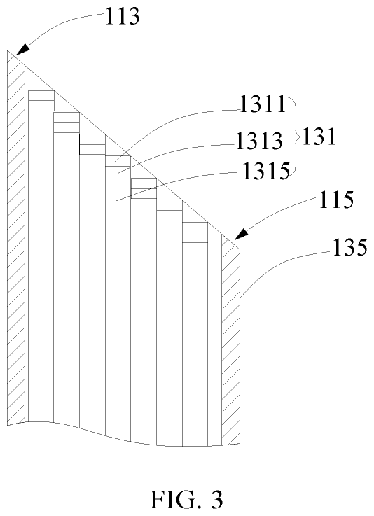

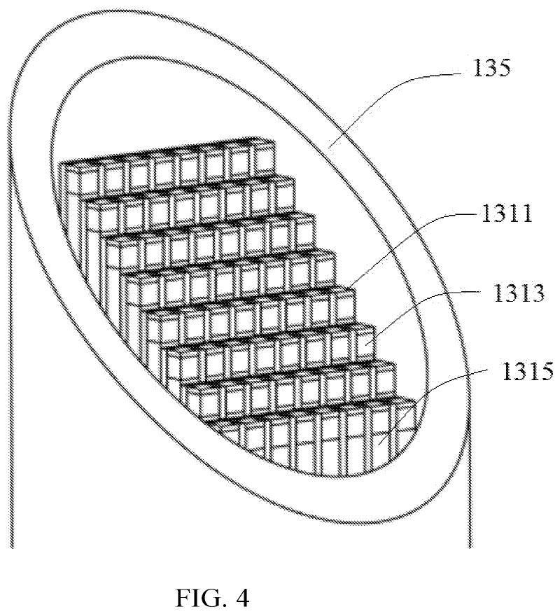

Referring to FIGS. 3 and 4 in conjunction, the puncture needle head 111 may have a pointed shape and have a containing head, and the plurality of ultrasonic array elements 131 may be arranged in the containing head. The puncture needle head 111 also has a needle tip side 113 and a needle tail side 115 opposite to each other, and the plurality of ultrasonic array elements 131 are arranged between the needle tip side 113 and the needle tail side 115. Furthermore, the ultrasonic array element 131 near the needle tip side 113 is higher than the ultrasonic array element 131 near the needle tail side 115. In particular, the height of the plurality of ultrasonic array elements 131 may gradually decrease in the direction away from the needle tip side 113, thus reducing the degree of obstruction of the ultrasonic array elements 131 by the edge of the tube shell 135, so that each ultrasonic array element 131 can transmit and receive ultrasonic waves to and from the outside in a wide range.

It should be noted that the pointed shape mentioned in this embodiment refers to the conventional needle tip shape. Of course, the puncture needle head 111 and the puncture head may also have other alternative shapes that can also achieve the puncture effect.

In this embodiment, the plurality of ultrasonic array elements 131 are distributed in an array, and an insulating barrier is arranged between every two adjacent ultrasonic array elements 131. In particular, the ultrasonic transducer 130 in this embodiment is a stepped array transducer, namely the plurality of ultrasonic array elements 131 are distributed in steps, and the height difference between every two adjacent ultrasonic array elements 131 is equal. That is, the ultrasonic array elements 131 of each row of the array are arranged by a gradient, and the height difference between two adjacent rows of ultrasonic array elements 131 is equal everywhere. Furthermore, the height and spacing are matched with the angle of the needle tube, and every two adjacent ultrasonic array elements 131 are isolated by insulating materials, such as plastic.

In this embodiment, the plurality of ultrasonic array elements 131 are all installed to orient towards the front of the puncture needle head 111. Of course, the plurality of ultrasonic array elements 131 may also be installed in other orientations slanted to the front of the puncture needle head 111. Alternatively, the plurality of ultrasonic array elements 131 may also be installed independently to assume their own orientations, and as long as the orientations in which the plurality of ultrasonic array elements 131 are installed can achieve the effect of transmitting and receiving ultrasonic waves to and from the front of the puncture needle head 111, they shall all fall in the scope of protection of the present disclosure.

In this embodiment, the end surface of the puncture needle head 111 has a puncture slope, and the outer edge of each ultrasonic array element 131 is flush with the puncture slope. Of course, the outer edge of each ultrasonic array element 131 may also have a certain small angle with the puncture slope. In particular, the top surface of each ultrasonic array element 131 forms a step surface of the stepped structure, the outer side of each ultrasonic array element 131 is the outer side of the step surface, and the puncture slope is the slope where the slope end surface of the puncture needle head 111 lies. The outer side of each ultrasonic array element 131 is coplanar with the end face of the puncture needle head 111, so that the slope of the step structure formed by the plurality of ultrasonic array elements 131 is in line with the slope of the puncture slope, which reduces the obstruction of the ultrasonic array elements 131 by the outer edge of the puncture needle head 111, so that each ultrasonic array element 131 can transmit and receive ultrasonic waves to and from the outside in a wide range. Furthermore, each ultrasonic array element 131 does not protrude from the puncture slope, which avoids excessive contact of the ultrasonic array element 131 with the body tissue during the puncture process, guarantees the transmitting and receiving range of each ultrasonic array element 131 to the greatest extent, thereby ultimately further improving the imaging effect.

In this embodiment, each ultrasonic array element 131 includes a matching layer 1311, a piezoelectric layer 1313, and a backing layer 1315, where the number of each layer is not limited. The backing layer 1315 is connected to the flexible member 133, the piezoelectric layer 1313 is arranged on the backing layer 1315, and the matching layer 1311 is arranged on the piezoelectric layer 1313. The outer side of the matching layer 1311 of each ultrasonic array element 131 is coplanar with the puncture bevel to prevent the ultrasonic array element 131 from protruding from the puncture needle head 111.

In view of the above, this embodiment provides an ultrasound guided puncture device 100, where the ultrasonic transducer 130 arranged in steps are installed on the puncture needle head 111 at the front end of the puncture needle tube 110. During the puncture process, multiple ultrasound array elements 131 are used to perform electronic scanning imaging to select the optimal puncture route and accurately reach the puncture area during the puncture process. After puncture guidance, the target area is anesthetized or the tissue and body fluid samples are obtained from the target area. The present disclosure performs electronic scanning imaging by using an interventional ultrasonic transducer that arranged in an array and has a stepped arrangement, which can obtain more information, have a wide imaging range, and can effectively distinguish the components and morphologies of the tissues in front of the needle tube. Furthermore, it is closer to the target tissue, the imaging is clearer, and it is not easy to be blocked by other parts to affect the imaging, and the operation is convenient and does not require the coordination of multiple medical staff.

### Second Embodiment

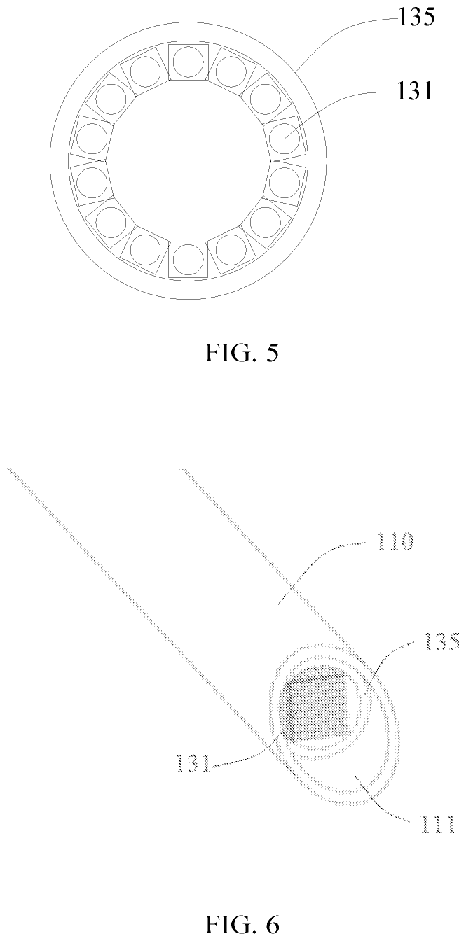

Referring to FIG. 5, this embodiment provides an ultrasound guided puncture device 100, the basic structure and principles of which and the technical effect produced whereby are the same as those of the first embodiment. For brevity of description, for parts not mentioned in this embodiment, turn to the corresponding content in the first embodiment.

This embodiment provides an ultrasound guided puncture device 100, including a puncture needle tube 110 and an ultrasound transducer 130. The puncture needle tube 110 has a puncture needle head 111 at one end, and the ultrasonic transducer 130 is accommodated in the puncture needle tube 110 and extends to the puncture needle head 111.

The ultrasonic transducer 130 includes a flexible member 133, a tubular housing 135, and a plurality of ultrasonic array elements 131 for transmitting and receiving ultrasonic waves, and are arranged at the puncture needle head 111. The tubular housing 135 is housed in the puncture needle tube 110 and extends to the puncture needle head 111. The flexible member 133 is housed in the tubular housing 135. The plurality of ultrasonic array elements 131 are arranged at one end of the flexible member 133 adjacent to the puncture needle head 111, and the plurality ultrasonic array elements 131 are all electrically connected to the flexible member 133. In particular, the bottom of each ultrasonic array element 131 may be provided with an electrode, and the electrodes of the plurality of ultrasonic array elements 131 may be led out through the flexible member 133 to facilitate electrical connection with other external control devices.

In this embodiment, the plurality of ultrasonic array elements 131 are annularly arranged in the tubular housing 135. In particular, the plurality of ultrasonic array elements 131 are arranged around the inside of the tubular housing 135, and the plurality of ultrasonic array elements 131 are arranged in the same plane. Of course, the plurality of ultrasonic array elements 131 may also decrease in height in succession along the direction from the needle tip side 113 to the needle tail side 115 of the puncture needle head 111, so as to reduce to a certain extent the shielding effect of the edge of the puncture needle head 111.

In this embodiment, the tubular housing 135 is retracted within the puncture needle head 111, and the plurality of ultrasonic array elements 131 are flush with the end surface of the tubular housing 135.

### Third Embodiment

Referring to FIG. 6, this embodiment provides an ultrasound guided puncture device 100, the basic structure and principles of which and the technical effect produced whereby are the same as those of the first embodiment. For brevity of description, for parts not mentioned in this embodiment, turn to the corresponding content in the first embodiment.

This embodiment provides an ultrasound guided puncture device 100, including a puncture needle tube 110 and an ultrasound transducer 130. The puncture needle tube 110 has a puncture needle head 111 at one end, and the ultrasonic transducer 130 is accommodated in the puncture needle tube 110 and extends to the puncture needle head 111.

The ultrasonic transducer 130 includes a flexible member 133, a tubular housing 135, and a plurality of ultrasonic array elements 131 for transmitting and receiving ultrasonic waves, and are arranged at the puncture needle head 111. The tubular housing 135 is housed in the puncture needle tube 110 and extends to the puncture needle head 111. The flexible member 133 is housed in the tubular housing 135. The plurality of ultrasonic array elements 131 are arranged at one end of the flexible member 133 adjacent to the puncture needle head 111, and the plurality ultrasonic array elements 131 are all electrically connected to the flexible member 133. In particular, the bottom of each ultrasonic array element 131 may be provided with an electrode, and the electrodes of the plurality of ultrasonic array elements 131 may be led out through the flexible member 133 to facilitate electrical connection with other external control devices.

In this embodiment, the ultrasonic transducer 130 is an area array transducer, and the plurality of ultrasonic array elements 131 are arranged in the tubular housing 135 in the same planar array. In particular, the plurality of ultrasonic array elements 131 and the tubular housing 135 are all retracted within the puncture needle head 111, and the plurality of ultrasonic array elements 131 are all flush with the end surface of the tubular housing 135.

### Fourth Embodiment

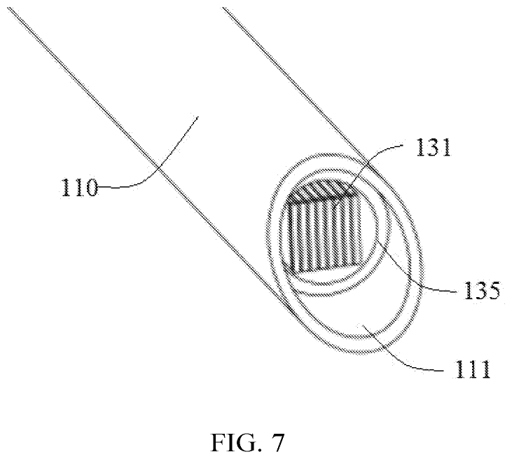

Referring to FIG. 7, this embodiment provides an ultrasound guided puncture device 100, the basic structure and principles of which and the technical effect produced whereby are the same as those of the first embodiment. For brevity of description, for parts not mentioned in this embodiment, turn to the corresponding content in the first embodiment.

This embodiment provides an ultrasound guided puncture device 100, including a puncture needle tube 110 and an ultrasound transducer 130. The puncture needle tube 110 has a puncture needle head 111 at one end, and the ultrasonic transducer 130 is accommodated in the puncture needle tube 110 and extends to the puncture needle head 111.

The ultrasonic transducer 130 includes a flexible member 133, a tubular housing 135, and a plurality of ultrasonic array elements 131 for transmitting and receiving ultrasonic waves, and are arranged at the puncture needle head 111. The tubular housing 135 is housed in the puncture needle tube 110 and extends to the puncture needle head 111. The flexible member 133 is housed in the tubular housing 135. The plurality of ultrasonic array elements 131 are arranged at one end of the flexible member 133 adjacent to the puncture needle head 111, and the plurality ultrasonic array elements 131 are all electrically connected to the flexible member 133. In particular, the bottom of each ultrasonic array element 131 may be provided with an electrode, and the electrodes of the plurality of ultrasonic array elements 131 may be led out through the flexible member 133 to facilitate electrical connection with other external control devices.

In this embodiment, the ultrasonic transducer 130 is a linear array transducer, and each ultrasonic array element 131 is line shaped, and the plurality of ultrasonic array elements 131 are parallel to each other and arranged in the tubular housing 135 in parallel along the same horizontal plane. In particular, the plurality of ultrasonic array elements 131 and the tubular housing 135 are all retracted within the puncture needle head 111, and the plurality of ultrasonic array elements 131 are all flush with the end surface of the tubular housing 135.

### Fifth Embodiment

This embodiment provides an ultrasound guided puncture apparatus, including an ultrasound electronic system (not shown) and an ultrasound guided puncture device 100. The basic structure and principles of the ultrasound guided puncture device 100 and the technical effects produced thereby are the same as those of the first embodiment. For brevity of description, for parts not mentioned in this embodiment, turn to the corresponding content in the first embodiment.

The ultrasound guided puncture device 100 includes a puncture needle tube 110 and an ultrasound transducer 130. One end of the puncture needle tube 110 has a puncture needle head 111. The ultrasound transducer 130 is accommodated in the puncture needle tube 110 and extends to the puncture needle head 111, and the ultrasound transducer 130 includes a plurality of ultrasonic array elements 131 for transmitting and receiving ultrasonic waves, and the plurality of ultrasonic array elements 131 are arranged at the puncture needle head 111. The ultrasonic electronic system is electrically connected to the ultrasonic transducer 130 for exciting the plurality of ultrasonic array elements 131 and processing echo signals received by the ultrasonic transducer 130.

In this embodiment, the ultrasound electronic system includes a controller and an imaging device. The imaging device is electrically connected to the ultrasound transducer for performing image reconstruction corresponding to the anatomical tissue structure based on the echo signals so as to obtain an image of the tissue in front of the puncture needle head 111. The controller is electrically connected to the plurality of ultrasonic array elements 131 of the ultrasonic transducer 130, and the angles at which the plurality of ultrasonic array elements 131 emit and receive ultrasonic waves can be adjusted by the controller, so that the imaging range can be rotated at a certain angle. The imaging device is electrically to the controller for imaging.

It should be noted that the image created by the imaging device here may be a two-dimensional ultrasound image or a three-dimensional ultrasound image. Although this disclosure has been described with reference to the current illustrative embodiments, those having ordinary skill in the art will appreciate that the above illustrative embodiments are merely used to illustrate the present disclosure and rather than limit the scope of protection of the present disclosure. Any modification, equivalent replacement, improvement, etc. made within the spirit and principle scope of the disclosure shall be included in the scope of protection of the present disclosure.

What is claimed is:

1. An ultrasound guided puncture device, comprising a puncture needle tube and an ultrasonic transducer, wherein the puncture needle tube comprises a puncture needle head at one end, and the ultrasonic transducer is housed in the puncture needle tube and extends to the puncture needle head, wherein the ultrasonic transducer comprises a plurality of ultrasonic array elements configured for transmitting and receiving ultrasonic waves, and wherein the plurality of ultrasonic array elements are arranged at the puncture needle head.

2. The ultrasound guided puncture device as recited in claim 1, wherein the puncture needle head is of a pointed shape and comprises a containing head, and wherein the plurality of ultrasonic array elements are arranged in the containing head.

3. The ultrasound guided puncture device as recited in claim 2, wherein the puncture needle head comprises a needle tip side and a needle tail side which are opposite to each other, and wherein the plurality of the ultrasonic array elements are arranged between the needle tip side and the needle tail side.

4. The ultrasound guided puncture device as recited in claim 2, wherein the plurality of ultrasonic array elements are distributed in an array.

5. The ultrasound guided puncture device as recited in claim 2, wherein the plurality of ultrasonic array elements are distributed horizontally.

6. The ultrasound guided puncture device as recited in claim 4, wherein the plurality of ultrasonic array elements are distributed horizontally.

7. The ultrasound guided puncture device as recited in claim 2, wherein the plurality of ultrasonic array elements are distributed in steps, and there is a height difference between the ultrasonic array elements distributed along the direction of the steps.

8. The ultrasound guided puncture device as recited in claim 4, wherein the plurality of ultrasonic array elements are distributed in steps, and there is a height difference between the ultrasonic array elements distributed along the direction of the steps.

9. The ultrasound guided puncture device as recited in claim 2, wherein an end surface of the puncture needle head comprises a puncture bevel, and the array composed of the plurality of ultrasonic array elements has an array bevel that matches the puncture bevel.

10. The ultrasound guided puncture device as recited in claim 4, wherein an end surface of the puncture needle head comprises a puncture bevel, and the array composed of the plurality of ultrasonic array elements has an array bevel that matches the puncture bevel.

11. The ultrasound guided puncture device as recited in claim 1, wherein the ultrasonic transducer further comprises a flexible member and a tubular housing, wherein the tubular housing is accommodated in the puncture needle tube and extends to the puncture needle head, the flexible member is accommodated in the tubular housing, the plurality of ultrasonic array elements are arranged at one end of the flexible member adjacent to the puncture needle head, and the plurality of ultrasonic array elements are all electrically connected to the flexible member.

12. The ultrasound guided puncture device as recited in claim 11, wherein the tubular housing comprises a puncture head at the end, which is accommodated in the puncture needle head, wherein an end surface of the puncture head is arranged flush or at an angle with an end surface of the puncture needle head.

13. The ultrasound guided puncture device as recited in claim 11, wherein the plurality of ultrasonic array elements are annularly arranged in the tubular housing.

14. An ultrasound guided puncture apparatus, comprising an ultrasound electronic system and the ultrasound guided puncture device as recited in claim 1, wherein the ultrasound electronic system is electrically connected to the ultrasound transducer and is configured for exciting the plurality of ultrasonic array elements and processing echo signals received by the ultrasonic transducer.

15. The ultrasound guided puncture apparatus as recited in claim 14, wherein the ultrasound electronic system comprises an imaging device, which is electrically connected to the ultrasound transducer and configured for reconstructing an image of an anatomical tissue structure based on the echo signals to obtain an image of a tissue in front of the puncture needle head.

16. The ultrasound guided puncture apparatus as recited in claim 14, wherein the puncture needle head is of a pointed shape and comprises a containing head, and wherein the plurality of ultrasonic array elements are arranged in the containing head.

17. The ultrasound guided puncture apparatus as recited in claim 16, wherein the puncture needle head comprises a needle tip side and a needle tail side which are opposite to each other, and wherein the plurality of the ultrasonic array elements are arranged between the needle tip side and the needle tail side.

18. The ultrasound guided puncture apparatus as recited in claim 16, wherein the plurality of ultrasonic array elements are distributed in an array.

19. The ultrasound guided puncture apparatus as recited in claim 16, wherein the plurality of ultrasonic array elements are distributed horizontally.

20. The ultrasound guided puncture apparatus as recited in claim 16, wherein the plurality of ultrasonic array elements are distributed in steps, and there is a height difference between the ultrasonic array elements distributed along the direction of the steps.
